(19) United States
(12) Patent Application Publication
(10) Pub. No.: US 2010/0124521 A1
(43) Pub. Date: May 20, 2010

(54) Full-Automatic Water-Bath Rotary Recuperated Autoclave with High Temperature & High Pressure

(76) Inventor: SHUWEN GUO, Weifang (CN)

Correspondence Address: GLOBAL IP SERVICES, 7285 W. Eagle Court, Winton, CA, 95388, US

(21) Appl. No.: 12/271,870

(22) Filed: Nov. 15, 2008

(51) Int. Cl. A61L 2/04

(52) U.S. Cl. 422/307

(57) ABSTRACT

A full-automatic water-bath rotary recuperated autoclave with high temperature & high pressure comprising a hot water tank, a process tank and its transmission system, characterized in that: Through bolt connection, said hot water tank is fixed on said process tank at the end of which a straight round end cover is provided; a holder for transmission system is mounted on the side of said straight round end cover; there is a bearing seat on the said holder; the main shaft is connected with the said bearing seat via the said bearing; the gears are fixed on the said main shaft and connected with the motor output wheels; the said motor is mounted on the said holder. Through above configuration, the present invention uses simple structural connection to solve effectively conveniently disadvantages of transmission system and improve operating life of the whole autoclave at the same time.

### FIELD OF THE INVENTION

The present invention relates to an autoclave used for sterilizing packaging food.

### BACKGROUND OF THE INVENTION

At present, transmission mode used for rotary cage of autoclave is chain transmission. Because there is cushion effect for tension of chain and chain itself, the stopping position of cage using chain transmission is not consistent stably. In transmission system, the end cover used to support the transmission system is convex hemisphere-shaped body. It is easy to deform when the autoclave is used in sterilization at high temperature. At the same time, the position of transmission mechanism is changed with deformation of end cover. Dislocation connection between the cage and transmission mechanism occurs easily so that rotation of the cage is not stable. For sealing of the end cover and transmission main shaft, packing seal is usually used at present. Water is easy to leak from such transmission seal during operation. It possesses short operating life and low tightness.

### SUMMARY OF THE INVENTION

Purpose of the present invention is to provide a new structure autoclave able to achieve effectively stable rotation of the cage and improve good sealing between transmission mechanism and the end cover and operating life of the whole transmission mechanism.

In accordance with the above purposes, a device according to the present invention comprises a hot tank, a process tank and its transmission system, characterized in that: Through bolt connection, said hot water tank is fixed on said process tank at the end of which a straight round end cover is provided; a holder for transmission system is mounted on the side of said straight round end cover; there is a bearing seat on the said holder; the main shaft is connected with the said bearing seat via the said bearing; the gears are fixed on the said main shaft and connected with the motor output wheels; the said motor is mounted on the said holder; mechanical seal is used at the joint of main shaft and straight round end cover.

Advantages of the present invention lie in: through above configuration, the present invention uses simple structural connection to solve effectively conveniently disadvantages of transmission system and improve operating life of the whole autoclave at the same time.

### BRIEF DESCRIPTION OF THE DRAWINGS

FIG. 1 is the abridged general view of the whole structure according to the present invention.

FIG. 2 is the structural enlargement view of process tank according to the present invention.

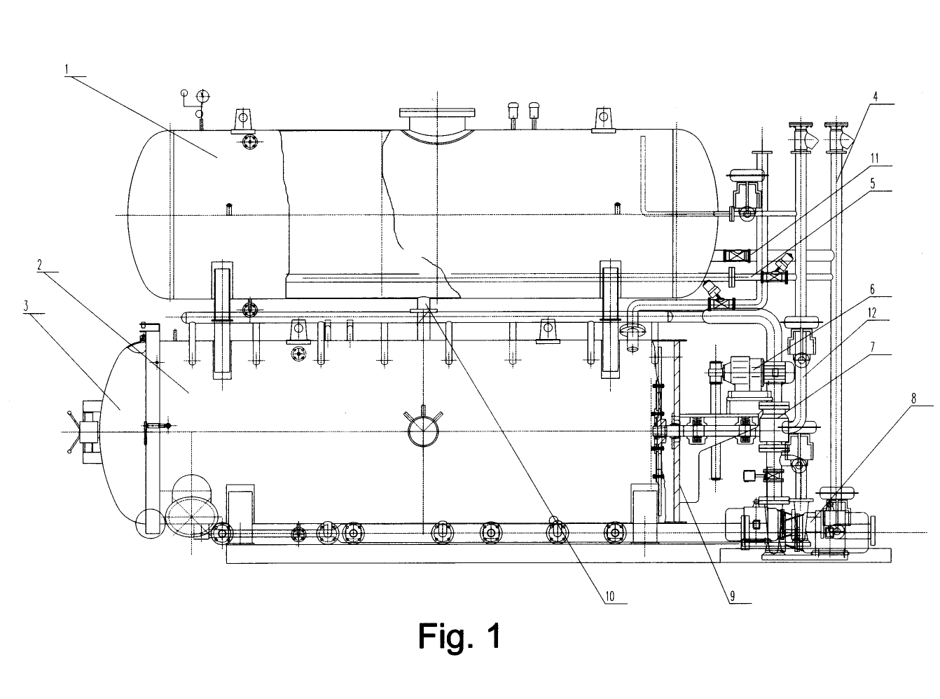

In FIG. 1, hot water tank 2, process tank 3, autoclave door 4, cool water pipe 5, steam pipe of hot water tank 6, transmission system 7, vapor & water mixer 8, cool water pump 9, straight round end cover 10, water drains 11, cool water valve 12, steam pipe 6-1 motor 6-2 mechanical seal 6-3 transmission gear 6-4 bearing seat 6-5 main shaft 6-6 cage 6-7 holder

### DETAILED DESCRIPTION OF THE INVENTION

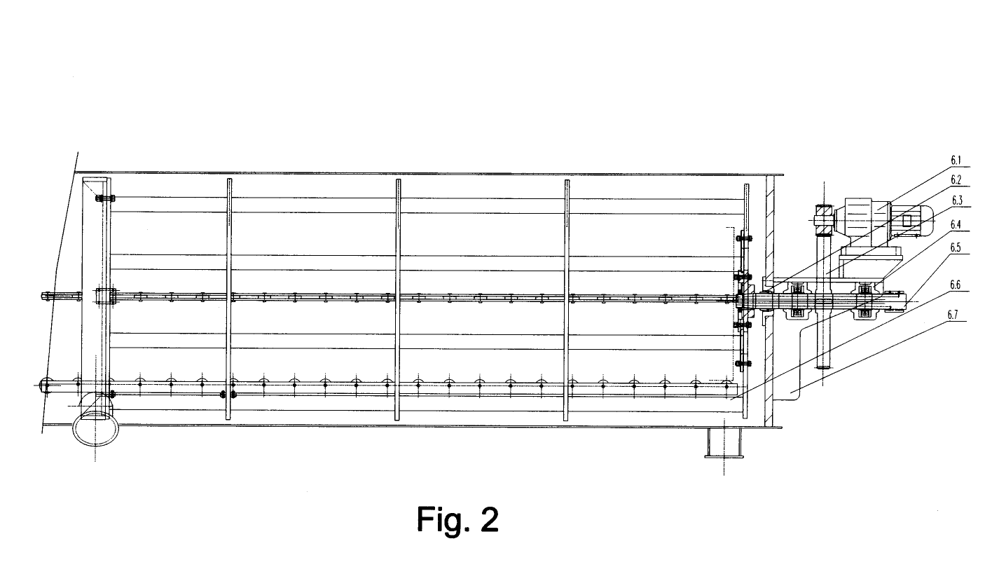

With reference to FIGS. 1 and 2, the present invention comprising a hot water tank 1, a process tank 2 and its transmission system 6, characterized in that: Through bolt connection, said hot water tank 1 is fixed on said process tank 2 at the end of which a straight round end cover 9 is provided; a holder 6-7 for transmission system is mounted on the side of said straight round end cover 9; there is a bearing seat 6-4 on the said holder 6-7; the main shaft 6-5 is connected with the said bearing seat 6-4 via the said bearing; the gears 6-3 are fixed on the said main shaft 6-5 and connected with the motor output wheels; the said motor 6-1 is mounted on the said holder 6-7; mechanical seal is used at the joint of main shaft 6-5 and straight round end cover 9.

The working process of the present invention is described in detail by reference to attached drawings: firstly, open cool water valve 11 to inject cool water into hot water tank 1; then open steam pipe 5 of hot water tank to heat cool water in hot water tank 1; after certain temperature is reached, autoclave door of process tank 2 is opened and food to be disinfected is put into the process tank. After autoclave 3 is sealed, water drains are opened and food in process tank 2 is disinfected by hot water in hot water tank 1. At the same time, the cage 6-6 is rotated under the action of main shaft 6-5 to achieve sterilization. Through steam pipe 12, vapor provided by vapor & water mixer is injected into process tank 2 to heat water in process tank 2 circularly. After sterilization is kept for certain time, it is ended. Hot water in process tank 2 is pumped back into process tank 2. Then take out of sterilized food. The whole sterilization is finished.

What is claimed is:

1. A full-automatic water-bath rotary recuperated autoclave with high temperature & high pressure comprising a hot water tank, a process tank and its transmission system, characterized in that: through bolt connection said hot water tank is fixed on said process tank, a straight round end cover is provided at the end of said process tank; a holder for the transmission system is mounted on the side of said straight round end cover; there is a bearing seat on said holder; a main shaft is connected with said bearing seat via bearings; gears are fixed on said main shaft and connected with motor output wheels; a motor is mounted on the said holder.
